(19) United States
(12) Patent Application Publication
(10) Pub. No.: US 2007/0073154 A1
(43) Pub. Date: Mar. 29, 2007

(54) Ultrasonic probe and ultrasonic diagnostic apparatus

(75) Inventor: Hiroyuki Karasawa, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/518,272

(22) Filed: Sep. 11, 2006

(30) Foreign Application Priority Data
Sep. 9, 2005 (JP) 2005-262363

(51) Int. Cl. A61B 8/14

(52) U.S. Cl. 600/459

(57) ABSTRACT

An ultrasonic diagnostic apparatus is provided with an ultrasonic probe that has at its end a low frequency ultrasonic transducer group and a high frequency ultrasonic transducer group, and a selection switch for selecting one of these transducer groups. According to the selection at the selection switch, a switching device of the ultrasonic probe connects either the low frequency ultrasonic transducer group or the high frequency ultrasonic transducer group to a multiplexer. The multiplexer selects from among ultrasonic transducers of the selected group ones to be driven at the same time, while shifting the driven transducers of the same group step by step.

### FIELD OF THE INVENTION

The present invention relates an ultrasonic probe that is provided with plural kinds of ultrasonic transducer groups, each group consists of a plurality of ultrasonic transducers having the same frequency bandwidth. The present invention also relates to an ultrasonic diagnostic apparatus using the ultrasonic probe.

### BACKGROUND OF THE INVENTION

Recently, medical diagnoses utilizing ultrasonic images have been put into practice in the medical field. To take an ultrasonic image, ultrasonic waves are radiated from an ultrasonic probe toward a certain region of a living body, and thereafter an echo signal is detected electrically from the living body. Based on the echo signal, the ultrasonic image is produced and displayed on an ultrasonic observer that is connected to the ultrasonic probe through connecters.

Scanning a region with ultrasonic waves can take an ultrasonotomographic image. Ultrasonic probes of mechanical scanning type turn or swing mechanically an ultrasonic transducer that sends and receives the ultrasonic waves. Ultrasonic probes of electronic scanning type have an array of ultrasonic transducers, which are switched on and off by electronic switches, to drive selected ones of these transducers.

However, in order to obtain adequate ultrasonotomographic images for exact medical diagnoses, it is necessary to adjust frequency of the ultrasonic waves in accordance with the depth of region to be scanned, because there are differences between individual living bodies, e.g. a difference in fat amount, and a difference in ultrasound propagation characteristics between tissues. In addition to that, high frequency ultrasonic waves can provide high-resolution images but cannot provide images of deep site. To cope with this problem, there is a method wherein low frequency ultrasonic waves are first radiated to observe a rough image of the whole region including the deep sites, and then high frequency ultrasonic waves are radiated to observe details of a suspected lesion on a high-resolution image.

For this purpose, a plurality of ultrasonic endoscopes or ultrasonic probes, which are loaded with ultrasonic transducers of different frequency bands, have conventionally been prepared, so that the operator can use some of them according to the site to be observed. Since it is necessary for intra-cavity diagnosis to introduce the ultrasonic probe through a channel of forceps into the patient body, the use of plural kinds of ultrasonic probes has been a main factor of increasing the load on the patient. To solve this problem, an ultrasonic probe has been suggested in Japanese Laid-open Patent Application No. 2005-103193, which has different kinds of ultrasonic transducer groups, each group consisting of a plurality of ultrasonic transducers of the same frequency band. According to this prior art, the single ultrasonic probe can radiate ultrasonic waves of different frequency bands.

More specifically, the ultrasonic probe of this prior art has a laminated structure having at least two layers of ultrasonic transducers, each layer consisting of a plurality of electric/ultrasound transducer elements. The individual layers are driven independently at the different frequencies. So the ultrasonic probe of this prior art is usable both for transcranial ultrasound Doppler monitoring, using a frequency of about 2 MHz, and for thrombolytic treatment, using a frequency of about 500 MHz.

Japanese Laid-open Patent Application No. 2003-169800 also suggests an ultrasonic probe having a plurality of ultrasonic transducers, which are arranged in a plane and consist of groups of different frequency characteristic curves from each other. The ultrasonic probe of the latter prior art widens the usable frequency bandwidth and thus facilitates modulations, such as harmonic imaging for producing adequate three-dimensional ultrasonic images.

However, the ultrasonic probe of the former prior art is automatically switched over between the transcranial ultrasound Doppler monitoring mode and the thrombolytic treatment mode at constant time intervals. The ultrasonic probe of the latter prior art just connects one or both of the two kinds of ultrasonic transducer groups to a sender section for sending a drive signal to excite the ultrasonic transducers, or a receiver section for receiving an echo signal from the living body. That is, the combinations of sending and receiving signals are fixed, so the operator cannot switch or select the frequency band of the ultrasonic wave. Accordingly, the prior arts cannot satisfy the demand of the operators for selecting the frequency of ultrasonic wave from the ultrasonic probe appropriately in accordance with required image resolution and depth of observation, which vary depending upon the diagnostic purpose.

Furthermore, according to the above prior arts, the number of wires to the ultrasonic transducers increases with the number of kinds of ultrasonic transducers. Correspondingly, the diameters of the ultrasonic probes of the prior arts come to be larger than a conventional one using a single kind of ultrasonic transducers. So the load on the patients at the time of introducing the ultrasonic probe into a body cavity gets heavier.

### SUMMARY OF THE INVENTION

In view of the foregoing, a primary object of the present invention is to provide an ultrasonic probe, which allows the operator to change the frequency of ultrasonic wave promptly in accordance with the intension of the operator without the need for replacing the probe, or without enlarging the diameter of the probe. Another object is to provide an ultrasonic diagnostic apparatus using the inventive ultrasonic probe.

According to the present invention, in an ultrasonic probe and an ultrasonic diagnostic apparatus using the ultrasonic probe, a plural kinds of groups of ultrasonic transducers are arranged at an end of the ultrasonic probe for radiating ultrasonic waves toward a living body and receiving echo signal from the living body, each group consisting of a plurality of ultrasonic transducers having the same frequency band that is different from another group, and one group to use is selected from among the plural kinds of groups in accordance with a control device that is entered by operating a control device.

According to a preferred embodiment, the ultrasonic probe further comprises signal lines for sending drive signals to the ultrasonic transducers and transmitting the echo signals received from the living body; and a switching device for switching connections to the signal lines between the plural kinds of groups in accordance with the control signal.

Preferably, the ultrasonic probe further comprises multiplexers for selecting ones to be driven at the same time from among the ultrasonic transducers of the one group as selected through the control device.

The different frequency bands of the plural kinds of groups of ultrasonic transducers are preferably set to cover a bandwidth from about 3 MHz to 20 MHz.

It is preferable to short-circuit the ultrasonic transducers of not-selected ones of the groups electrically.

Because the control device is operated to enter the control signal for selecting one group from among the plural kinds of groups of ultrasonic transducers, the operator can change the frequency band of the ultrasonic wave appropriately at any timing.

The switching device, which switches connections to the signal lines between the plural kinds of groups of ultrasonic transducers in accordance with the operation on the control device, contributes to reducing the requisite number of wirings. The multiplexers, which select ones to be driven at the same time from among the ultrasonic transducers of the one group as selected through the control device, also contributes to reducing the requisite number of wirings. Thus, the ultrasonic probe of the present invention may have a smaller diameter even while it can radiate ultrasonic waves of different frequency bands.

### BRIEF DESCRIPTION OF THE DRAWINGS

The above and other objects and advantages of the present invention will be more apparent from the following detailed description of the preferred embodiments when read in connection with the accompanied drawings, wherein like reference numerals designate like or corresponding parts throughout the several views, and wherein:

FIG. 1 is a block diagram illustrating schematic structure of an ultrasonic diagnostic apparatus according to an embodiment of the invention;

FIG. 2 is a pattern diagram for explaining aperture width, focal length, focal position and beam diameter of an ultrasonic transducer;

FIG. 3A is an enlarged sectional view of a low frequency ultrasonic transducer group at an end portion of an ultrasonic probe;

FIG. 3B is an enlarged sectional view of a high frequency ultrasonic transducer group at an end portion of the ultrasonic probe;

FIG. 4 is a block diagram illustrating schematic structure of an ultrasonic probe according to another embodiment of the invention;

FIG. 5 is a perspective view, partly in section, of an ultrasonic probe according to a further embodiment of the invention; and

FIG. 6 is a top plan view illustrating an end portion of an ultrasonic probe according to another embodiment of the invention.

### DESCRIPTION OF THE PREFERRED EMBODIMENTS

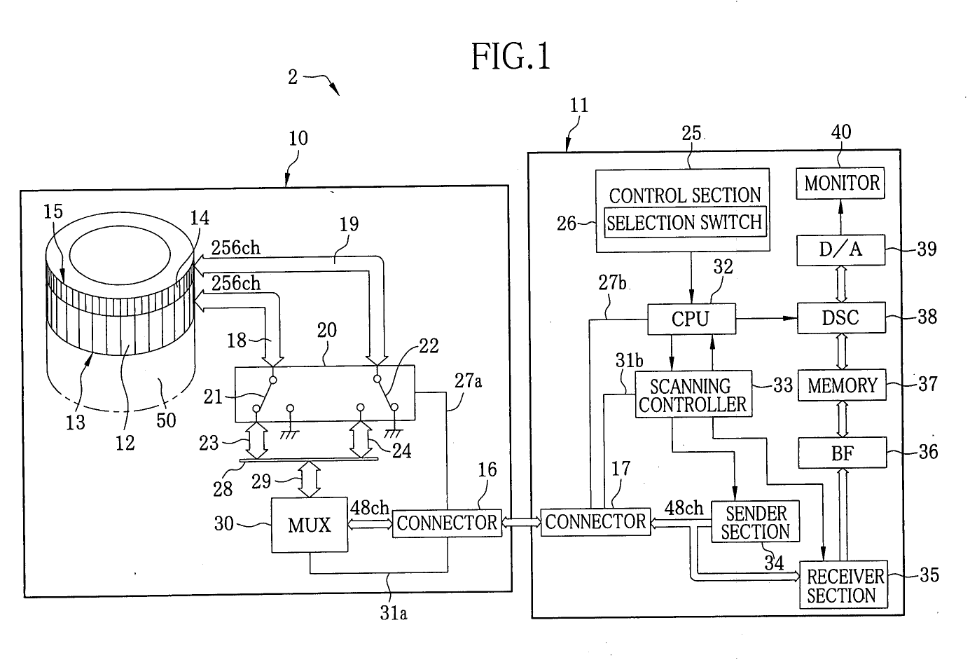

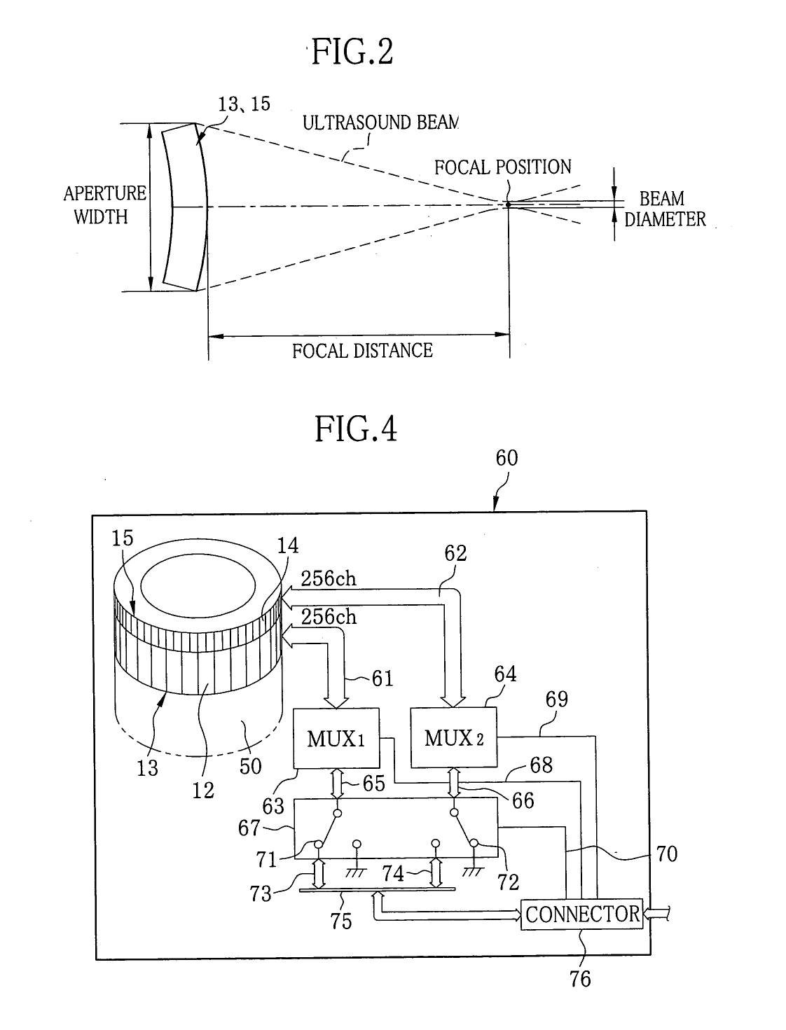

FIG. 1 shows an ultrasonic diagnostic apparatus 2 of the present invention, which consists of an ultrasonic probe 10 and an ultrasonic observer 11. The ultrasonic probe 10 is provided with a low frequency ultrasonic transducer group 13 consisting of 256 low frequency ultrasonic transducers 12, and a high frequency ultrasonic transducer group 15 consisting of 256 high frequency ultrasonic transducers 14. The low frequency ultrasonic transducers 12 have an effective frequency band of 3.5 MHz to 8 MHz, whereas the high frequency ultrasonic transducers 14 have an effective frequency band of 8 MHz to 20 MHz. These transducer groups 13 and 15 are disposed at an end portion of a sheath of the ultrasonic probe 10, the sheath being omitted from the drawings but made of a soft flexible material.

The ultrasonic transducers 12 and 14 have such properties as shown in TABLE 1, which permit executing diagnoses with the ultrasonic probe 10 while adjusting its ultrasonic frequency according to the purpose and application. Concretely, for the purpose of observing the whole image including deeper portions, the low frequency ultrasonic wave of 4 MHz or 7.5 MHz is usable. On the other hand, for the purpose of observing details of a suspected site of disease, the high frequency ultrasonic wave of 12 MHz or 20 MHz is usable. Note that “beam diameter” in TABLE 1 represents a diameter of an ultrasonic beam at a focal position, which is obtained when the aperture width and the focal length are set at the values shown in TABLE 1. “Depth” represents a depth of an observed site of a living body, at which an optimum image is obtainable when the frequency is set at the value shown in TABLE 1.

TABLE 1

| | | FOCAL | | BEAM |
|---|---|---|---|---|
| FREQUENCY | APERTURE | LENGTH | DEPTH | DIAMETER |
| (MHz) | WIDTH (mm) | (cm) | (cm) | (mm) |
| 4 | 6.4 | 8 | 6 or more | 4.8 |
| 7.5 | 4.8 | 4 | 6 or less | 1.7 |
| 12 | 3.2 | 2 | 3 | 0.8 |
| 20 | 2.4 | 0.5 | 1 or less | 0.16 |

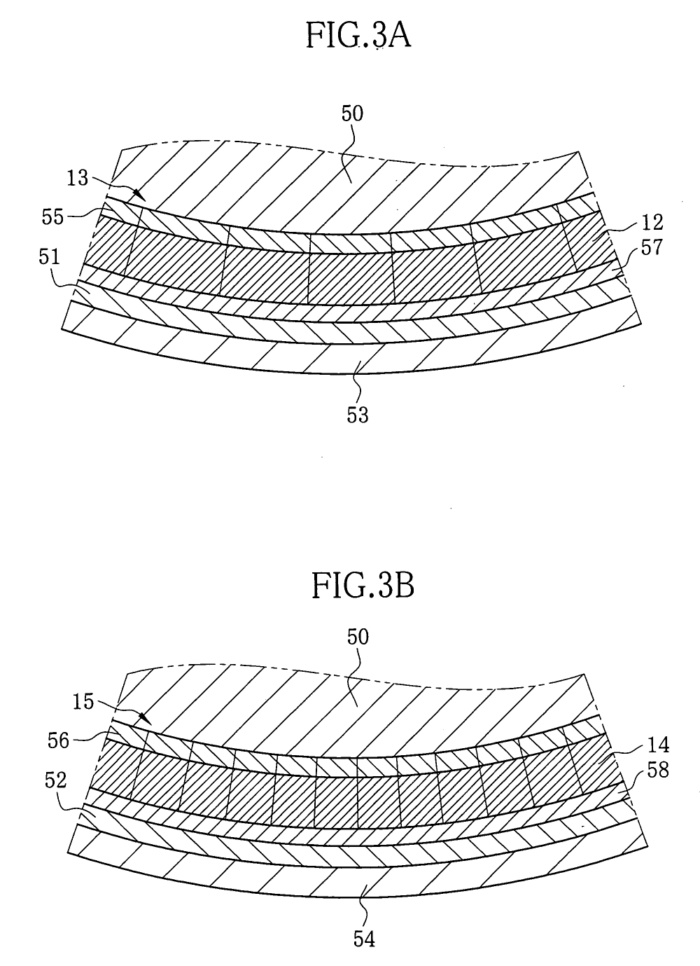

As shown in FIG. 3, the ultrasonic probe 10 is a so-called radial electronic scanning type, wherein the low frequency ultrasonic transducer group 13 and the high frequency ultrasonic transducer group 15 are arranged around a cylindrical backing material 50, and an acoustic matching layer 51 or 52 and an acoustic lens 53 or 54 are laminated atop another on the transducer group 13 or 15, respectively. The ultrasonic probe 10 is used as a fine probe that is introduced into an opening for forceps of an electronic endoscope, or as an ultrasonic endoscope that is integrated with an electronic endoscope. So the ultrasonic probe 10 is inserted in a cavity of a living body when it is used.

The ultrasonic transducers 12 and 14 are located between individual electrodes 55 and 56, on one hand, and common electrodes 57 and 58, on the other hand, respectively. The common electrodes 57 and 58 are grounded. The ultrasonic transducers 12 and 14 and the individual electrodes 55 and 56 are isolated from each other with a filling material that is not shown but implied by solid lines.

A voltage applied across from the individual electrodes 55 or 56 to the common electrode 57 or 58 excites the low or high frequency ultrasonic transducers 12 or 14 to oscillate, respectively. Thereby, the low or high frequency ultrasonic transducers 12 or 14 emit the ultrasonic wave toward the internal body site to be observed, and receive echo signals from the internal body site, to generate a voltage corresponding to the echo signals.

On scanning the ultrasonic wave across the internal body site to be observed, the ultrasonic transducers 12 and 14 are driven block by block. Each block consists of neighboring forty-eight pieces of the ultrasonic transducers 12 or 14, and the forty-eight transducers of one block are driven at the same time. On receiving the echo signals from the observed site, the transducers of one block receive the echo signals simultaneously. Each time drive signals are sent to excite the low or high frequency ultrasonic transducers 12 or 14, and the echo signals are received, the neighboring 48 ultrasonic transducers 12 or 14 to be driven at one time are shifted step by step, i.e. by one or several pieces. Thus, the ultrasonic transducers 12 or 14 to send the drive signals and receive the echo signals are switched over from one another. Note that the number of ultrasonic transducers to send and receive the signals is not necessarily equal. In that case, the control is carried out based on the bigger number of ultrasonic transducers of one block.

Referring back to FIG. 1, the ultrasonic probe 10 is connected to the ultrasonic observer 11 by plugging a connector 16 of the ultrasonic probe 10 into a connector 17 of the ultrasonic observer 11. The connector 16 is provided at a trailing end of a not-shown code that extends from the sheath.

The individual electrodes 55 and 56 of the ultrasonic transducers 12 and 14 are connected to one ends of first and second signal lines 18 and 19, respectively. The first and second signal lines 18 and 19 provide 256 channels each, and are connected at the other ends to a switching device 20. The switching device 20 consists of first switches 21 for 256 channels and second switches 22 for 256 channels, so the first signal lines 18 are connected to the first switches 21, and the second signal lines 19 are connected the second switches 22, in one-to-one relationships. The first and second switches 21 and 22 are made of semiconductor switching elements, such as MOSFETs.

The first and second switches 21 and 22 are connected at other ends to third and fourth signal lines 23 and 24 and the earth. In an initial position, the first switches 21 are connected to the third signal lines 23, while the second switches 22 are connected to the earth. By operating a selection switch 26, which is provided in an operating section 25 of the ultrasonic observer 11, the first switches 21 are switched over between the third signal lines 23 and the earth, and the second switches 22 are switched over between the earth and the fourth signal lines 24 in cooperation with the first switches 21. So the first switches 21 are connected to the third signal lines 23 while the second switches 22 are grounded and electrically short-circuited. While the second switches 22 are connected to the fourth signal lines 24, the first switches 21 are grounded and electrically short-circuited.

The switching device 20 is connected to first control lines 27a and 27b that lead through the connectors 16 and 17 to a CPU 32 of the ultrasonic observer 11. Each time the switching device 20 receives a first control signal of 1-bit from the CPU 32 through the first control lines 27a and 27b, the switching device 20 switches the first and second switches 21 and 22 in the above-described manner.

The third and fourth signal lines 23 and 24 are connected to a switching port 28, which is connected to fifth signal lines 29. Corresponding to the selection by the selection switch 26, the switching port 28 connects either the third signal lines 23 or the fourth signal lines 24 to the fifth signal lines 29.

The fifth signal lines 29 are connected to a multiplexer (MUX) 30. The multiplexer 30 switches the fifth signal lines 29 to select from among the fifth signal lines 29 ones to be used for transmitting the drive signals and the received echo signals. Each time the drive signals are sent to the 48 transducers of one block, and the echo signals are received, the 48 transducers to constitute one block are shifted by one or several pieces among the 256 ultrasonic transducers 12 or 14 of the selected group 13 or 15.

The multiplexer 30 are connected to second control lines 31a and 31b that leads through the connectors 16 and 17 to a scanning controller 33 of the ultrasonic observer 11. Upon receipt of a second control signal of 8-bits from the scanning controller 33 through the second control lines 31a and 31b, the multiplexer 30 switches the fifth signal lines 29 in the above described manner. Note that the multiplexer 30 and the switching device 20 are built in a hollow inside the backing material 50.

The ultrasonic observer 11 is totally controlled by the CPU 32. The CPU 32 is connected to the scanning controller 33, which is connected to a sender section 34 and a receiver section 35, to send reference pulses for controlling operations of these sections.

Under the control of the scanning controller 33, the sender section 34 sends the drive signals of 48 channels to those ultrasonic transducers 12 or 14 selected by the multiplexer 30, while the receiver section 35 receives the echo signals of 48 channels from the site to be observed, which are taken by the ultrasonic transducers 12 or 14 selected by the multiplexer 30. The receiver section 35 processes the echo signals for sensitivity time control (STC), whereby sensitivity is adjusted to time corresponding to propagation distance (depth) of the ultrasonic wave. The scanning controller 33 switches over the timing of sending the drive signals from the sender section 34 and the timing of receiving the echo signals on the receiver section 35.

The echo signals received on the sender section 35 are input in a beam former (BF) 36. The beam former 36 delays the 48-channel echo signals by a given time to adjust phases of all the echo signals into alignment with each other. Thereafter the beam former 36 sums up the echo signals.

After the echo signals are phase-aligned and summed in the beam former 36 and digitalized, the digital echo signal is stored in a memory 37. Under the control of the CPU 32, a digital scan converter (DSC) 38 reads out the digital echo signal from the memory 37 and converts it into an NTSC signal as a TV scanning signal. A digital/analog converter 39 converts the NTSC signal into an analog signal. The analog signal is sent from the D/A converter 39 to a monitor 40 for displaying an ultrasonic image.

The control section 25 is formed as a control panel that is provided with various operation buttons including the selection switch 26. The CPU 32 controls the respective sections of the ultrasonic diagnostic apparatus 2 in response to operational signals entered through the control section 25.

Now the operation of the ultrasonographic apparatus 2 will be described. First, the ultrasonic probe 10 is inserted into a body cavity till it reaches a site to be observed, and the control section 25 is operated to unfreeze. Then the sender section 34 sends out the 48-channel drive signals under the control of the scanning controller 33. In this initial position, the first switches 21 are connected to the third signal lines 23, whereas the second switches 22 are grounded. Accordingly, the low frequency ultrasonic transducers 12 are driven by the drive signals.

The drive signals from the sender section 34 are fed through the connectors 16 and 17 to the multiplexer 30. The multiplexer 30 selects forty-eight lines for sending the drive signals from among the fifth signal lines 29, based on the second control signal sent from the scanning controller 33 through the second control lines 31a and 31b. So the drive signals from the sender section 34 are sent through the fifth signal lines 29, the third signal lines 23, the first switches 21 and the first signal lines 18 to a designated block of the low frequency ultrasonic transducers 12. Then the forty-eight ultrasonic transducers 12 of the designated block are excited by the drive signals to emit the low frequency ultrasonic wave toward the site to be observed.

After the drive signals are sent, the scanning controller 33 switches the sending operation of the sender section 34 to the receiving operation of the receiver section 35. Then, the echo signals taken by the forty-eight low frequency ultrasonic transducers 12 of the designated block are fed through the first signal liens 18, the first switches 21, the third signal lines 23, the fifth signal liens 29, the multiplexer 30 and the connectors 16 and 17 to the receiver section 35.

The receiver section 35 subjects the input echo signals to the STC processing. Thereafter, the echo signals are phase-aligned and summed in the beam former 36, and are stored as a digital signal in the memory 37. Thereafter, based on the second control signal, the multiplexer 30 shifts those to be driven among the low frequency ultrasonic transducers 12, by one or several pieces, till the same process as above is executed on the last block of the low frequency ultrasonic transducers 12.

When the scanning is accomplished with the 256 low frequency ultrasonic transducers 12 in this way, the digital scan converter 38 reads out the digital echo signals stored in the memory 37 and converts them into the NTSC signals. The NTSC signals are converted back to the analog signal through the D/A converter 39, and are displayed as an ultrasonic image on the monitor 40. The above-described sequential operation is repeated cyclically till a freeze command is given by operating the control section 25.

Thereafter when the selection switch 26 is operated to cause the CPU 32 to send the first control signal through the first control lines 27a and 27b to the switching device 20, the first switches 21 are switched from the third signal lines 23 to the earth side, whereas the second switches 22 are switched to the fourth signal lines 24. Thereby, the drive signal from the sender section 34 are sent through the connectors 16 and 17, the multiplexer 30, the fifth signal lines 29, the fourth signal lines 24, the second switches 22 and the second signal lines 19 to a designated block of the high frequency ultrasonic transducers 14.

The echo signals taken from the internal body site by these high frequency ultrasonic transducers 14 are sent through the second signal lines 19, the second switches 22, the fourth signal lines 24, the fifth signal lines 29, the multiplexer 30 and the connectors 16 and 17 to the receiver section 35. Thereafter, the same process as described with respect to the low frequency ultrasonic transducers 12 is carried out, to display an ultrasonic image from the high frequency ultrasonic transducers 14 on the monitor 40.

As described so far, the ultrasonic diagnostic apparatus 2 is provided with the selection switch 26 for selecting between the low frequency ultrasonic transducer group 13 and the high frequency ultrasonic transducer group 15. So the operator can change the used frequency band of the ultrasonic wave according to the site to be observed.

Furthermore, the ultrasonic probe 10 is provided with the switching device 20 and the multiplexer 30. The switching device 20 switches over the connection of the first and second signal lines 18 and 19 in response to the first control signal from the selection switch 26, to drive either the low frequency ultrasonic transducer group 13 or the high frequency ultrasonic transducer group 15. The multiplexer 30 switches the fifth signal lines 29 to select the ultrasonic transducers to be driven at the same time from among the ultrasonic transducers 12 or 14. Thanks to the switching device and the multiplexer 30, the requisite number of channels of the signal lines is reduced to 48, though 512 channels would otherwise be necessary for the transducer groups 13 and 15. So the ultrasonic probe 10 can have a smaller diameter. Because being used for intra-cavity diagnosis, the ultrasonic probe 10 of a smaller diameter is effective especially for reducing the load on the patient.

Since the low and high frequency ultrasonic transducer groups 13 and 15 provide a frequency bandwidth ranging from about 3 MHz to 20 MHz, the ultrasonic diagnostic apparatus 2 of the present invention can produce ultrasonic images whose range of depth and resolution satisfy necessity for medical diagnoses, without the need for changing probes.

Since the first switches 21 are grounded alternately with the second switches 22, while the other switches 22 or 21 are respectively connected to the signal lines 24 or 23, unused one of the ultrasonic transducer groups is electrically short-circuited. Therefore, the ultrasonic transducers of the unused group do not unnecessarily oscillate to generate a noise, which damages quality of the subsequent ultrasonic image. That is, the present invention prevents image deterioration resulted from the noise caused by the unnecessary oscillation of the unused transducers.

FIG. 4 shows an ultrasonic probe 60 according to another embodiment of the present invention, wherein low frequency transducers 12 are connected through first signal lines 61 of 256 channels to first multiplexer (MUX1) 63, whereas high frequency transducers 14 are connected through second signal lines 62 of 256 channels to second multiplexer (MUX2) 64 respectively. The first and second multiplexers 63 and 64 are connected through third and fourth signal lines 65 and 66, each having 48 channels, to a switching device 67, respectively.

The ultrasonic probe 60 may be used in combination with the ultrasonic observer 11. Respectively based on second and third 8-bit control signals, which are sent from the scanning controller 33 through second and third control lines 68 and 69, the first and second multiplexers 63 and 64 select from among the first and second signal lines 61 and 62 those signal lines to be used for sending the drive signals and receiving the echo signals. Also, based on the first control signal of 1-bit that is sent from the CPU 32 through a first control line 70, the switching device 67 switches over first and second switches 71 and 72 in the same way as in the above-described embodiment. So either fifth signal lines 73 or sixth signal lines 77 are connected to the multiplexer 63 or 64, to use ultrasonic transducer groups 13 and 15 alternatively. Reference numerals 75 and 76 designate a switching port and a connector 76 respectively.

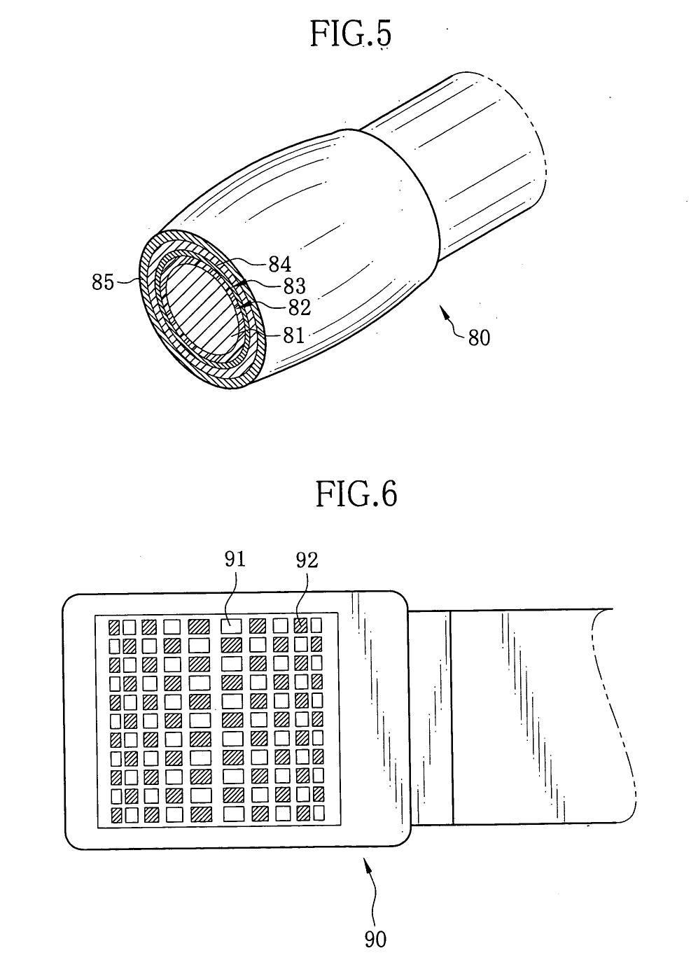

The present invention is not limited to the ultrasonic probes 10 and 60 as illustrated in the above embodiments. As shown for example in an ultrasonic probe 80 of FIG. 5, it is possible to arrange a low frequency transducer group 82 around a cylindrical backing material 81, and laminate a high frequency transducer group 83 on the low frequency transducer group 82. Reference numerals 84 and 85 designate an acoustic matching layer and an acoustic lens respectively.

The present invention is not to be limited to the radial electronic scanning type ultrasonic probe, like the probes 10 and 80, but also applicable to a convex electronic scanning type probe, e.g. an ultrasonic probe 90 as shown in FIG. 6. In the convex electronic scanning type ultrasonic probe 90, low frequency transducers 91 that are illustrated as blank quadrangles, and high frequency transducers 92 that are illustrated as hatched quadrangles are arranged alternately in a two-dimensional array.

Although the selection switch 26 is provided in the control section 25 of the ultrasonic observer 11, a selection switch for selecting between the low frequency ultrasonic transducer group and the high frequency ultrasonic transducer group may be provided in a control section of the ultrasonic probe, together with an air sending button and a watering button.

It is to be noted that the number of ultrasonic transducers of one group, the number of ultrasonic transducers of one block to be driven at the same time, and the number of groups of the ultrasonic transducers are not limited to the above embodiments, but may be modified appropriately according to the specification of the ultrasonic diagnostic apparatus.

Thus, the present invention is not to be limited to the above embodiments but, on the contrary, various modifications will be possible without departing from the scope of claims appended hereto.

What is claimed is:

1. An ultrasonic probe comprising:
a plural kinds of groups of ultrasonic transducers arranged at an end of said ultrasonic probe for radiating ultrasonic waves toward a living body and receiving echo signal from the living body, each group consisting of a plurality of ultrasonic transducers having the same frequency band that is different from another group; and
a control device operated to enter a control signal for selecting one group to use from among said plural kinds of groups.

2. An ultrasonic probe as claimed in claim 1, further comprising:
signal lines for sending drive signals to said ultrasonic transducers and transmitting the echo signals received from the living body; and
a switching device for switching connections to said signal lines between said plural kinds of groups in accordance with said control signal.

3. An ultrasonic probe as claimed in claim 1, further comprising multiplexers for selecting ones to be driven at the same time from among the ultrasonic transducers of the one group as selected through said control device.

4. An ultrasonic probe as claimed in claim 1, wherein said ultrasonic probe is introduced into a cavity of the living body to use for intra-cavity diagnoses.

5. An ultrasonic probe as claimed in claim 1, wherein the different frequency bands of said plural kinds of groups of ultrasonic transducers are set to cover a bandwidth from about 3 MHz to 20 MHz.

6. An ultrasonic probe as claimed in claim 1, wherein the ultrasonic transducers of not-selected ones of said groups are electrically short-circuited.

7. An ultrasonic diagnostic apparatus using an ultrasonic probe, comprising:
a plural kinds of groups of ultrasonic transducers disposed at an end of said ultrasonic probe for radiating ultrasonic waves toward a living body and receiving echo signal from the living body, each group consisting of a plurality of ultrasonic transducers having the same frequency band that is different from another group; and
a control device operated to enter a control signal for selecting one group to use from among said plural kinds of groups.

8. An ultrasonic diagnostic apparatus as claimed in claim 7, wherein said ultrasonic probe comprises signal lines for sending drive signals to said ultrasonic transducers and transmitting the echo signals received from the living body, and a switching device for switching connections to said signal lines between said groups in accordance with said control signal, so that said drive signals are sent only to the ultrasonic transducers of the one group as selected through said control device.

9. An ultrasonic diagnostic apparatus as claimed in claim 7, wherein said ultrasonic probe comprises multiplexers for selecting ones to be driven at the same time from among the ultrasonic transducers of the one group as selected through said control device.

10. An ultrasonic diagnostic apparatus as claimed in claim 7, wherein said ultrasonic probe is introduced into a cavity of the living body to use for intra-cavity diagnoses.

11. An ultrasonic diagnostic apparatus as claimed in claim 7, wherein the different frequency bands of said plural kinds of groups of ultrasonic transducers are set to cover a bandwidth from about 3 MHz to 20 MHz.

12. An ultrasonic diagnostic apparatus as claimed in claim 7, wherein the ultrasonic transducers of not-selected ones of said groups are electrically short-circuited.

13. An ultrasonic diagnostic apparatus as claimed in claim 7, further comprising an ultrasonic observer having a device for displaying an ultrasonic image based on said echo signal, said ultrasonic observer sending said drive signals to and receiving said echo signals from said ultrasonic probe.

14. An ultrasonic diagnostic apparatus as claimed in claim 9, wherein said ultrasonic transducers are driven block by block in one group, each block consisting of a limited number of ultrasonic transducers, and said multiplexers select the block to drive, and wherein said drive signals and said echo signals are transmitted in channels of the limited number.
